(19) United States
(12) Patent Application Publication
(10) Pub. No.: US 2025/0275826 A1
(43) Pub. Date: Sep. 4, 2025

(54) ADJUSTABLE DEPTH STOP SYSTEM FOR SURGICAL ACCESSORY

(71) Applicant: ClearPoint Neuro, Inc., Solana Beach, CA (US)

(72) Inventors: Nathan Williams, Solana Beach, CA (US); Roberto F. Rubio, Solana Beach, CA (US); Maxwell J. Daly, Solana Beach, CA (US)

(21) Appl. No.: 18/857,761

(22) PCT Filed: Apr. 18, 2023
(86) PCT No.: PCT/US2023/018959

§ 371 Date: Oct. 17, 2024

Related U.S. Application Data
(60) Provisional application No. 63/332,441, filed on Apr. 19, 2022.

(51) Int. Cl. A61B 90/00

(52) U.S. Cl. CPC A61B 90/08; A61B 90/03; A61B 2090/033; A61B 2090/0811

(57) ABSTRACT

A depth stop system can be mechanically coupled to a surgical device for controlling the orientation and/or surgical depth of the surgical device into anatomy. Some non-limiting examples of surgical devices are side-firing laser catheters, biopsy needles, MER probes, deep brain stimulation (DBS) leads and radially branching cannulas.

### CROSS-REFERENCE TO RELATED APPLICATION

This application claims priority to U.S. Patent Application No. 63/332,441, filed Apr. 19, 2022, entitled “ADJUSTABLE DEPTH STOP SYSTEM FOR SURGICAL ACCESSORY”, the contents of which are hereby incorporated by reference herein its entirety.

### BACKGROUND

Fixtures device or tools are often used to control the precise insertion of a surgical device (e.g., deep brain stimulation (DBS) electrodes, MER probes, laser catheters and biopsy needles) into the human or animal anatomy such as into a brain. The insertion orientation and insertion depth are specific considerations taken into account when inserting a surgical device into the anatomy via the fixture device. In this regard, it is desirable for a user to accurately control fine adjustments of the insertion orientation and/or depth of the surgical device via the fixture device.

Disclosed herein is a fixture device that can be used to control insertion depth and/or orientation of a surgical device into the anatomy.

### SUMMARY

Disclosed is a depth stop system that can be mechanically coupled to a surgical device for controlling the orientation and/or surgical depth of the surgical device into anatomy. The type of surgical device can vary and can be any surgical device that is insertable into anatomy.

In one aspect, there is disclosed a device for adjusting a position of a surgical device relative to anatomy, the device comprising: a base portion; a depth slider portion movably attached to the base portion, wherein the depth slider portion releasably locks to the surgical device, the depth slider portion configured to move relative to the base portion along an axis; a gear raiser portion mechanically coupled to the depth slider portion and the base portion, wherein the gear raiser portion can be actuated to control movement of the depth slider portion along the axis; and an attachment mechanism configured to attach to a surgical frame.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1 shows a perspective view of an adjustable depth stop system.

FIG. 2 shows the depth stop system in partial transparent and cross section view.

FIG. 3 shows a schematic representation of the depth stop system mechanically coupled to a surgical frame.

FIGS. 4A-9 show another embodiment.

### DETAILED DESCRIPTION

Before the present subject matter is further described, it is to be understood that this subject matter described herein is not limited to particular embodiments described, as such may of course vary. It is also to be understood that the terminology used herein is for the purpose of describing particular embodiments only, and is not intended to be limiting. Unless defined otherwise, all technical terms used herein have the same meaning as commonly understood by one skilled in the art to which this subject matter belongs.

Disclosed is a depth stop system that can be mechanically coupled to a surgical device for controlling the orientation and/or surgical depth of the surgical device into anatomy. Some non-limiting examples of surgical devices are side-firing laser catheters, biopsy needles, MER probes, deep brain stimulation (DBS) leads and radially branching cannulas.

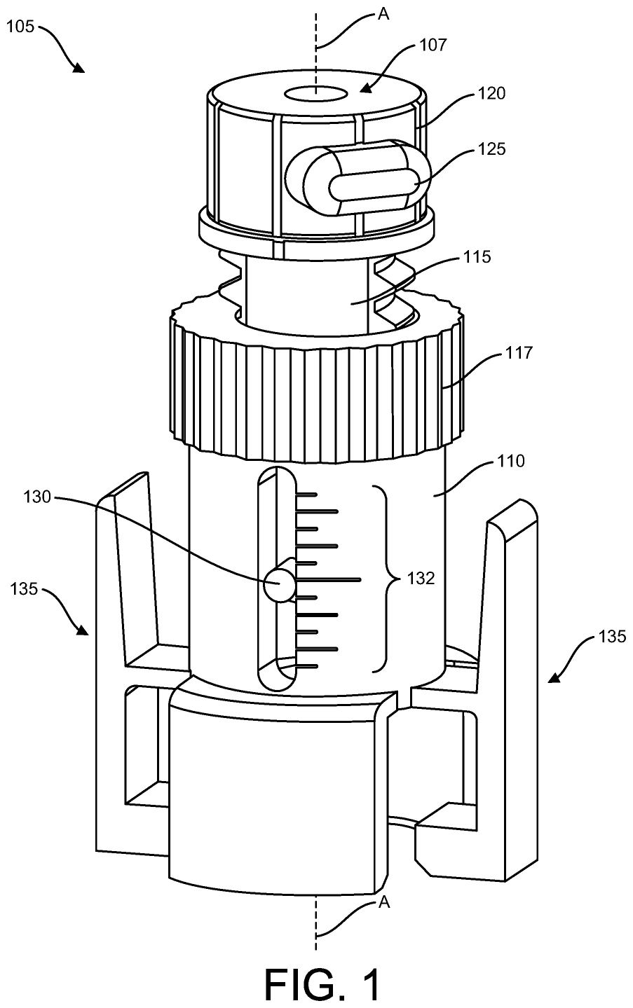

FIG. 1 shows a perspective view of depth stop system 105 that can be mechanically attached to a surgical device, such as by inserting the surgical device into a top opening 107 that communicates with an internal lumen of the depth stop system 105. The internal lumen also communicates with a bottom opening of the depth stop system 105 such that the surgical device can protrude out of the bottom end of the system 105 (and into anatomy when the depth stop system 105 is coupled to anatomy.) At least a first portion of the depth stop system 105 can be locked or otherwise immobilized relative to the surgical device. The first portion of the depth stop system 105 can be moved or otherwise adjusted relative to a second portion of the depth stop system 105 in a manner that allows adjustment of a position and/or orientation of the surgical device relative to anatomy while the second portion of the depth stop system stays fixed or immobilized relative to the anatomy, as described below.

With reference still to FIG. 1, the depth stop system 105 includes an outer housing or base 110 that is generally elongated and/or cylindrical in shape (although the shape may vary.) A depth slider 115 is movably positioned within or on the base 110 such that the depth slider 115 can move relative to the base 110 along an elongate axis A. The depth slider 115 can be an elongated body that forms and defines the internal lumen of the depth stop system 105 for receiving the surgical device. In this regard, the depth slider 115 has an internal lumen sized and shaped to receive an elongated surgical device therethrough. The depth slider 115 is movable within the base 110 such as by sliding within the base along the axis A, as described more fully below.

The depth slider 115 is coupled to a device lock 120 positioned at a top end (or other location) of the depth slider 115. The device lock 120 receives the surgical device and can be locked or immobilized relative to the surgical device, which inserts into the top opening 107 and into the internal lumen of the depth stop system 105. The depth slider 115 has an actuator such as a lock button 125 that can be actuated to control locking and release of the depth slider 115 relative to the surgical device when the surgical device is inserted into the device lock 120 and depth slider 115. The lock button 125 can be spring-loaded such that it directly or indirectly asserts a compression force against the surgical device when positioned within the lumen to hold the surgical device in place until the lock button 125 is depressed or otherwise actuated to release the surgical device. In an embodiment, the device lock 120 can rotate relative to the depth slider 115 about the axis A such that the surgical device (when attached and locked to the depth slider 115) can be locked to the device lock 120 but can also translate (such as along axis A) and rotate (such as about axis A) relative to the base 110 of the depth stop system 105. In another embodiment described below, the device lock may be removable from the rest of the assembly. This can make it easier to attach the device lock to the surgical device. In an embodiment, the device lock is prepackaged in a pre-assembled, attached state to the surgical device.

A gear raiser 117 is mechanically coupled to the depth slider 115 and the base 110 such as via a threaded connection. In an embodiment, the gear raiser 117 is a collar that positioned on the base 110 and that threadedly interacts with the depth slider 115. The gear raiser 117 can be actuated (such as via a rotational movement) to control movement of the depth slider 115 relative to the base 110, as described further below. A height indicator or height marker 130 is attached to the depth slider 115 so as to provide an indication of depth of the surgical device into the anatomy, as described further below. The height marker 130 can be a prong that extends through an elongated slot in the base 110. The height marker 130 can be visually measured against a depth scale 132 on the base 110 to provide an indication of depth, as described further below. The depth scale can be formed by indentations or markings along an outer surface of the base 110. In another embodiment described below, the depth scale is printed or otherwise positioned on the depth slider.

One or more attachment mechanisms 135 are attached to the base 110 and are configured to attach the depth stop system 105 to a surgical guide member, such as a surgical frame, that is fixedly secured to the anatomy. Thus, the depth stop system 105 attaches to a surgical guide member, which attaches to the patient's anatomy. The attachment mechanisms 135 can be one or more clips for example that are configured to removably clip onto the surgical guide. In another embodiment described below, the attachment mechanism includes one or more components, such as tabs, that mechanically couple to the surgical frame,

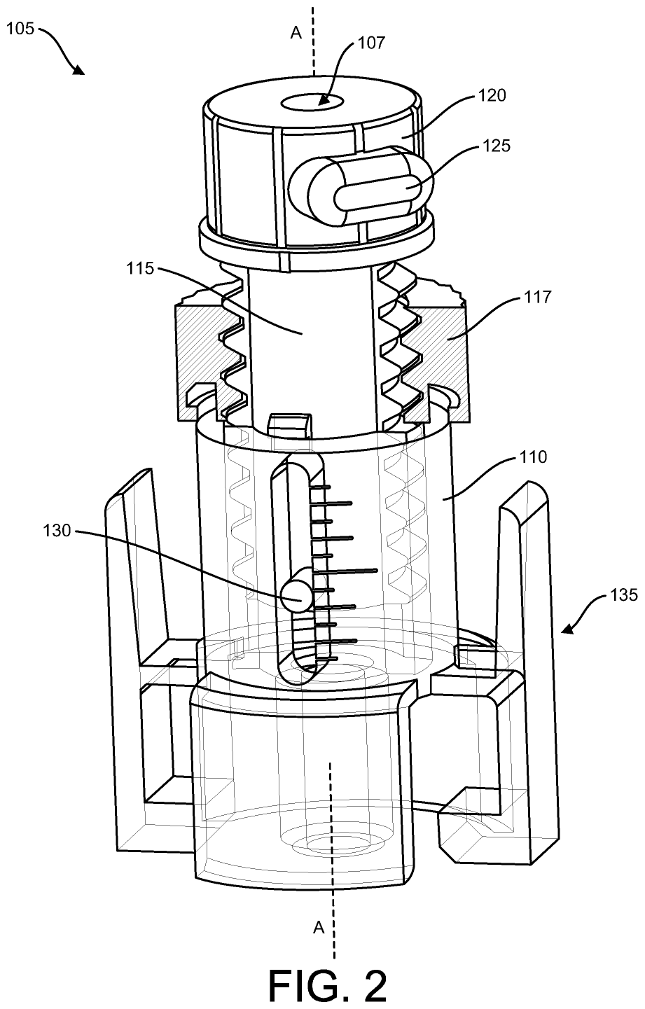

FIG. 2 shows the depth stop system 105 in a partial cross-section and transparent view. The depth slider 115 is an elongated body having an internal lumen that can be coaxial with the axis A. As mentioned, the internal lumen is sized and shaped to receive a surgical device therethrough. The depth slider 115 has a threaded outer surface with threads that threadedly coupled to internal threads of the gear raiser 117, which serves as an annular collar or nut that can be rotated relative to the depth slider 115 to cause movement or translation of the depth slider 115 along the axis A and relative to the base 110. As mentioned, the height marker 130 may extend through the slot in the base 110. The depth slider 115 and the base 110 can mechanically interact to prevent the depth slider 115 from rotating relative to the base. For example, the depth slider 115 can be keyed to prevent rotation of the depth slider 115 relative to the base 110. In this regard, the depth slider can have a key shaped member that mechanically interacts with the base to prevent relative rotation therebetween.

As mentioned, the device lock 120 can rotate relative to the depth slider 115. Such rotation can be unconstrained such that the device lock 120 freely rotates about a 360-degree range of rotation. Alternately, the rotation of the device lock 120 can be constrained, such as to have a limited maximum rotation angle. The rotational movement can be smooth and continuous, or it can incorporate stops at set angles (such as by clicking every 45 degrees). The device lock 120 and the depth slider 115 can incorporate complementary markings to indicate the angle of rotation. For example, the device lock 120 can have one or more markings that can be aligned with one or more markings on the depth slider 115 to provide an indication to a user as to an angle of rotation therebetween. In addition, a locking mechanism (such as a set screw or thumb knob) can be incorporated that allows the device lock 120 to be secured relative to the depth slider 115 such as to prevent unwanted rotation.

The depth stop system 105 can allow the surgical device to be initially loaded into the opening 107 at any angle (relative to the axis A) or could be keyed so the device must be loaded at a specific angle (relative to the axis A). For example, the inner diameter of the opening 107 or the top of the device lock 120 can have a notch that corresponds to a matching feature on the surgical device being loaded.

The depth stop system 105 is represented in FIGS. 1 and 2 as a separate, stand-alone structure configured to be mounted to a surgical fixture or frame, which is attached to anatomy. Alternatively, depth stop system 105 or any of its features can be incorporated directly into the fixture or frame. The system 105 may or may not be MR conditional. In an embodiment, the system is configured to mechanically nest within or on the frame.

As mentioned, the depth slider 115 has one or more external threads that mate with corresponding threads on the inner surface of the gear raiser 117. A user can actuate the gear raiser 117 (such as by rotating the gear raiser) to cause a mechanical interaction between the threads and thereby cause the depth slider 115 to move upward or downward relative to the base 110 along the axis A. As mentioned, a surgical apparatus can be fixedly attached to the device lock 120, which is also attached to the depth slider 115. Thus, movement of the depth slider 115 along the axis A results in corresponding movement of the surgical apparatus along the axis A. The configuration of the threads can be used to determine how much the depth slider 115 moves per rotation of the gear raiser 117.

The threads on the depth slider 115 can vary. For example, FIG. 2 shows the threads being positioned intermittently (i.e., on two opposing surfaces) on the outer surface of the depth slider 115. In an example variation, the depth slider 115 can have 3 or 4 sets of gear threads extending from its sides. Alternatively, the depth slider 115 can have threading that wraps around the entire perimeter except for a skived notch or notches that provide a basis for orientation.

The gear thread geometry can be customized to provide any amount of depth slider 115 movement per turn of the gear raiser 117. In a non-limiting example, the threading pitch can be 0.5 mm or 1.0 mm to move the slider 0.5 mm or 1.0 mm per revolution of the gear raiser 117. The threading can also be oriented so that turning the gear riser 117 clockwise moves the depth slider 115 up or moves the depth slider 115 down. The range of movement can be determined by the size of the depth slider 115 and the base 110. A maximum amount of depth slider length can be as long or as short as desired.

The illustrated embodiment shows the gear raiser 117 as configured to spin coaxially around the depth slider 115. Alternatively, another mechanical configuration such as worm gear can be mounted to the side of the base 110 to enable and control or actuation via a thumb knob. The depth slider 115 can also be configured to move freely up and down “by hand” until a lock is engaged to freeze it at a desired position relative to the base 110.

As mentioned, a user can monitor and gauge such movement by viewing movement of the height marker 130 relative to the depth scale 132.

Different mechanisms can be used to secure the surgical device into the depth stop system 105. For example, a set screw, compressed O-ring or chuck style clamp can be used. An embodiment described below uses a mechanical chuck to secure the surgical device to the system. In addition, soft grip materials such as a rubber gasket can be added to the push button or other device lock mechanism to increase tackiness or friction, prevent crush damage to the device and help prevent movement.

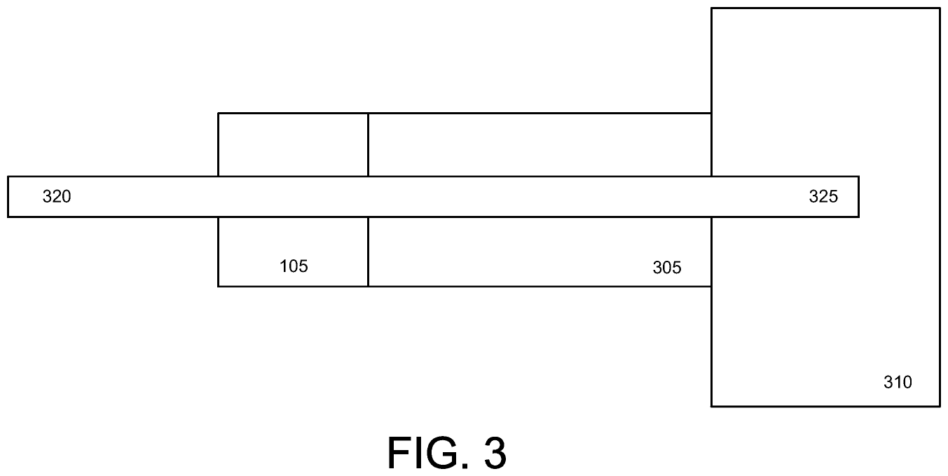

FIG. 3 shows a schematic representation of the depth stop system 105 mechanically coupled to a surgical frame 305. In use, the depth stop system 105 is secured to a surgical fixture or frame 305 such as via the clips of the attachment mechanism 135. Or, as mentioned, the depth stop system 105 can be incorporated into the surgical fixture or frame rather than being a separate structure. The surgical fixture or frame is secured to an anatomical structure such as a skull 310 for example. The user then inserts the surgical device 320 (such as a cannula) into the opening 107 (FIG. 1) and into the lumen of the depth stop system 105 so that a distal end 325 of the surgical device 320 protrudes out of the distal (or bottom) end of the depth stop system 105 while the proximal end of the surgical device protrudes out of the proximal (or top) end of the depth stop system 105.

The user can lock or immobilize the surgical device 320 relative to the depth slider 115 using the device lock 120. The user can adjust the position of the surgical device 320 therein by locking and releasing the surgical device using the button 125. With the surgical device locked to the device lock 120 and depth slider 115, the user can adjust the position of the surgical device by actuating the gear raiser to finely adjust the position of the depth slider 115 (and the attached surgical device) along axis A. The distal end of the surgical device is inserted into the anatomy and this permits the user to adjust the position, or depth, of the surgical device relative to the anatomy.

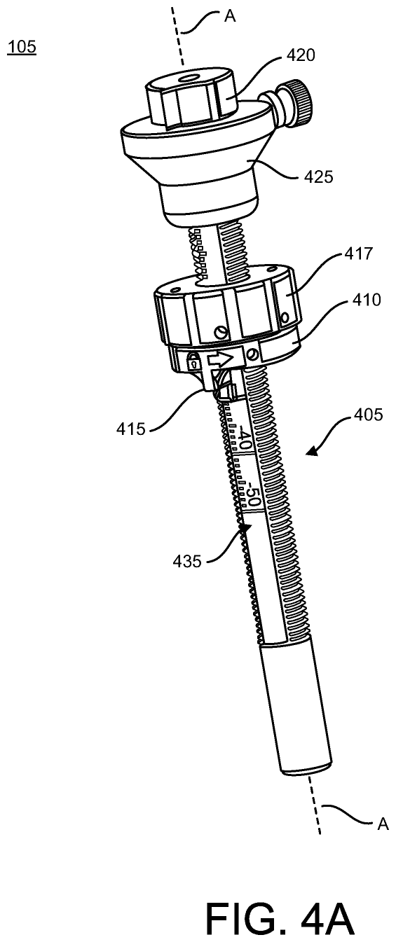

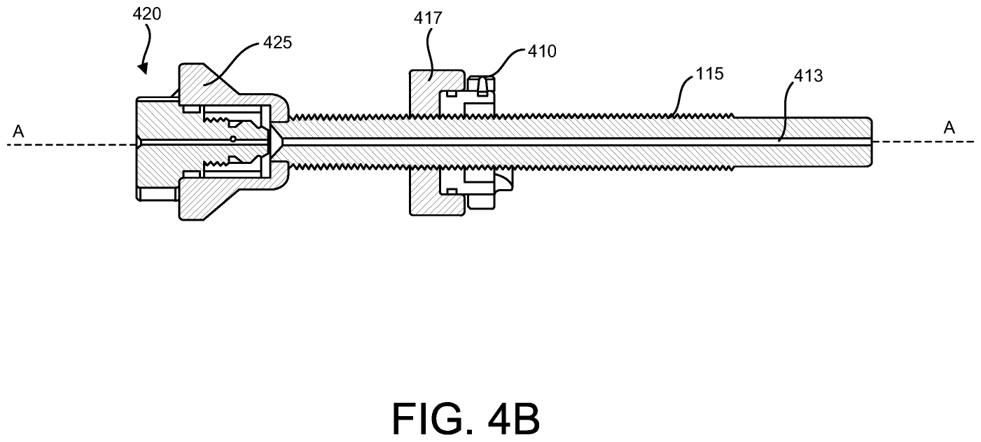

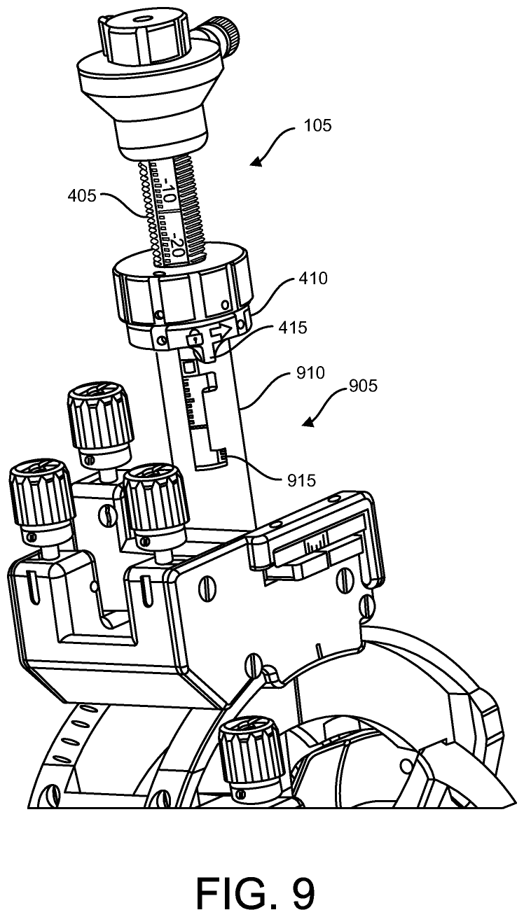

FIGS. 4A and 4B show another embodiment of the depth stop system 105, which includes an elongated depth slider 405 having an internal lumen 413 that extends along an axis A. A base 410 is rotatably coupled to the depth slider 405 and can be locked in position relative to the depth slider 405 and a surgical frame. The base 410 includes a tab 415 or other structure configured to mechanically couple to complementary-shaped structures on a surgical frame, as described below with reference to FIG. 9. A gear raiser 417 is mechanically coupled to the depth slider 405 (such as via a threaded connection) and to the base 410. The base 410 and gear raiser 417 have cylindrical collar shapes in FIG. 4 although the shapes can vary.

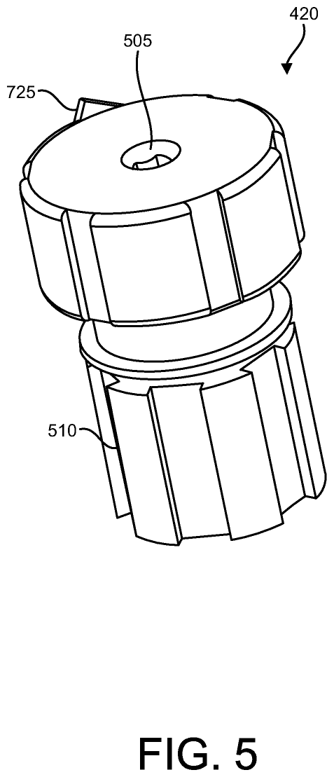

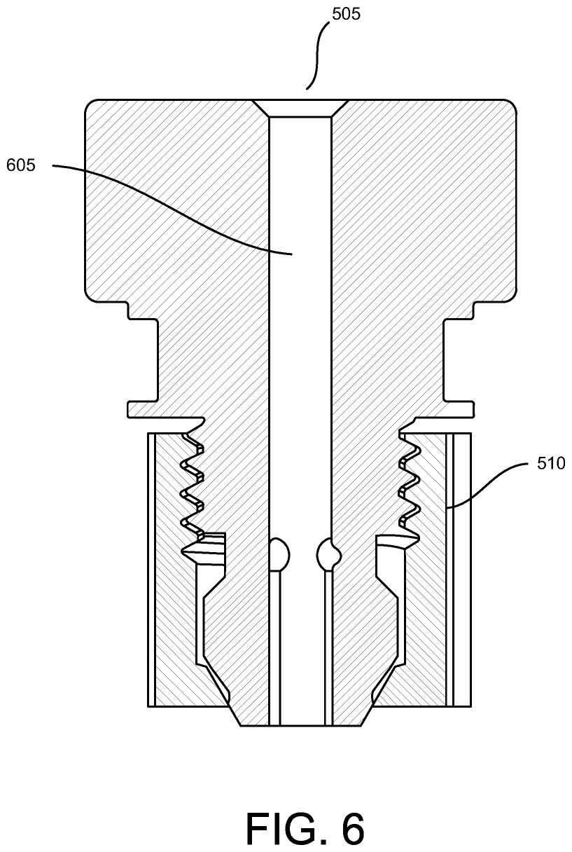

With reference still to FIGS. 4A and 4B, the system 105 includes a removable device lock 420 removably attached to the depth slider 405 such as by being removably insertable into a seat 425 positioned on the depth slider 405. The device lock 420 receives the surgical device and can be locked or immobilized relative to the surgical device. FIG. 5 shows the device lock 420 in a stand-alone state removed from the depth slider 405. The device lock 420 has a top opening 505 that receives the surgical device such that the surgical device can be inserted into a lumen of the device lock. The opening 505 communicates with an internal lumen 605 (FIG. 6) that communicates in a co-axial relationship with the internal lumen 413 (FIG. 4B) of the of the depth slider when the device lock 420 is in the depth slider. The device lock includes a locking sleeve 510 that can be rotated (or otherwise actuated) to lock or immobilize the surgical device to the device lock 420 when positioned therein. For example, the locking sleeve 510 of the device lock 420 can be rotated such that it causes the device lock 420 to pinch or constrain the surgical device mounted therein. FIG. 6 shows a cross-sectional view of the device lock 420.

With reference again to FIG. 5, the opening 505 can have a key-shape or other specific shape that mates with the surgical device only when the surgical device is in a predetermined orientation relative to the opening 505. In this manner, the surgical device must be inserted into the opening 505 when at the predetermined orientation such as in a key configuration. Thus, the device lock and the surgical device are both keyed to mate in only a specific manner or orientation.

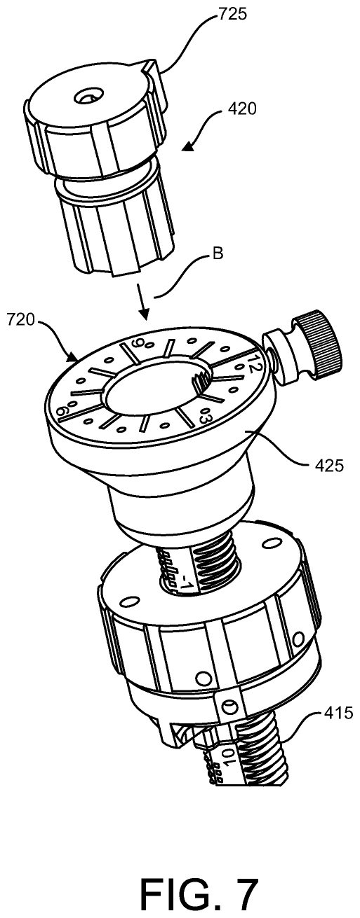

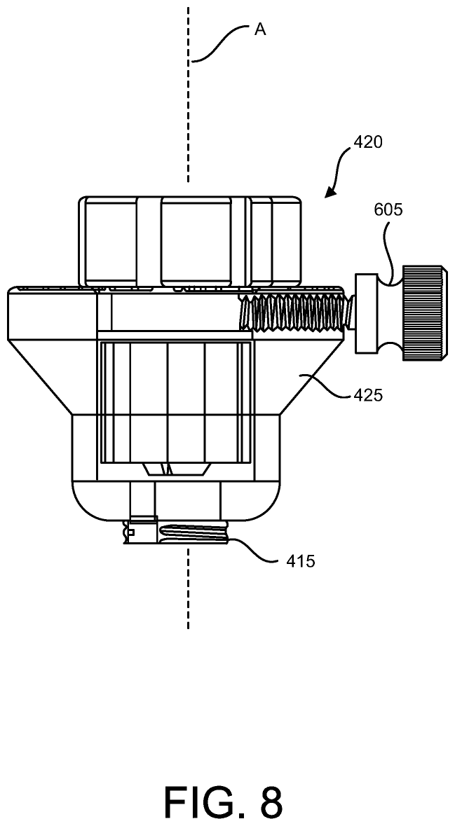

As mentioned, the device lock 420 can come prepackaged with or premounted on the surgical device. As shown in FIGS. 7 and 8, the device lock 420 inserts into the seat 425 of the depth slider 405 (as represented by arrow B in FIG. 7). A device such as a lock screw 805 (FIG. 8) or chuck mechanism can be used to lock or immobilize the device lock 420 to the seat 425 of the depth slider 405. Prior to being locked, the device lock 420 can rotate within the seat 425 about the axis A. The seat 425 can include a scale 720 (FIG. 7) or other indicator that provides a user with a visual indication of an angle of rotation of the device lock 420 relative to the seat 425. In this regard, the device lock 420 includes a corresponding angle indicator, such as a pointer 725 that interacts with the scale 720 to provide the visual indication.

FIG. 9 shows the depth stop system 105 mechanically coupled to or nested with a surgical frame 905. The elongated depth slider 405 slidably inserts into a complementary shaped lumen of a tower 910 of the surgical frame 905. As mentioned, the base 410 includes a tab 415 or other structure configured to mechanically couple to complementary-shaped structures on a surgical frame, such as to one or more slots 915 of the frame 905 to lock the depth stop system in place relative to the frame such as at any of a variety of depths relative to the frame. The depth slider 405 includes a height or depth scale (FIG. 4) 435 or other indicator that can provide a user with a visual indication of the depth level of the depth stop system 105 relative to the frame and/or anatomy.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

1. A device for adjusting a position of a surgical device relative to anatomy, the device comprising:
a base portion;
a depth slider portion movably attached to the base portion, wherein the depth slider portion releasably locks to the surgical device, the depth slider portion configured to move relative to the base portion along an axis;
a gear raiser portion mechanically coupled to the depth slider portion and the base portion, wherein the gear raiser portion can be actuated to control movement of the depth slider portion along the axis; and
an attachment mechanism configured to attach to a surgical frame.

2. The device of claim 1, further comprising a device lock attached to the depth slider, the device lock configured to lock to the surgical device to secure the surgical device to the depth slider.

3. The device of claim 2, wherein the device lock includes an actuator that can be actuated to lock and release the surgical device relative to the device lock.

4. The device of claim 3, wherein the actuator is a button.

5. The device of claim 3, wherein the actuator is a set screw or a chuck.

6. The device of claim 1, wherein the gear raiser portion has an internal thread that mates with an external thread of the base portion.

7. The device of claim 1, wherein the device is MR conditional.

8. The device of claim 1, wherein the gear raiser is a nut.

9. The device of claim 1, wherein the gear raiser is configured to spin co-axially around the base portion.

10. The device of claim 1, wherein the gear raiser comprises a worm gear.

11. The device of claim 1, wherein the depth slider has a height.

12. The device of claim 2, wherein the device lock is removable from the depth slider or other portion of the device.

13. The device of claim 2, wherein the device lock can rotate relative to the base when in an unlocked.

14. The device of claim 15, wherein the device lock includes an angle indicator.

15. The device of claim 1, wherein the device lock and the surgical device are both keyed to mate in only a specific manner or orientation.
